(19) United States
(12) Patent Application Publication
(10) Pub. No.: US 2022/0151474 A1
(43) Pub. Date: May 19, 2022

(54) MEDICAL IMAGE PROCESSING DEVICE AND MEDICAL OBSERVATION SYSTEM

(71) Applicant: Sony Olympus Medical Solutions Inc., Tokyo (JP)

(72) Inventors: Narumi SATO, Tokyo (JP); Koji KOJIMA, Tokyo (JP)

(73) Assignee: Sony Olympus Medical Solutions Inc., Tokyo (JP)

(21) Appl. No.: 17/396,784

(22) Filed: Aug. 9, 2021

(30) Foreign Application Priority Data
Nov. 18, 2020 (JP) 2020-191957

(51) Int. Cl. A61B 1/04; A61B 1/06; A61B 1/00

(52) U.S. Cl. CPC A61B 1/043; A61B 1/0684; A61B 1/00009; A61B 1/0646

(57) ABSTRACT

A medical image processing device includes: fluorescence image acquisition circuitry configured to acquire a fluorescence image; and a fluorescence image processor configured to execute image processing on the fluorescence image. A light receiving surface of the imaging unit is provided with a color filter in which red, green, and blue filter groups having spectral characteristics different from each other are arrayed in a specific format, the fluorescence image includes red component information, green component information, and blue component information corresponding to the spectral characteristics of the red, green, and blue filter groups, respectively, and the fluorescence image processor is configured to combine the respective red component information, green component information, and blue component information for each pixel of the fluorescence image in a state where a weight of the red component information is lowered as compared with weights of the green component information and the blue component information.

### CROSS-REFERENCE TO RELATED APPLICATION

This application claims priority from Japanese Application No. 2020-191957, filed on Nov. 18, 2020, the contents of which are incorporated by reference herein in its entirety.

### BACKGROUND

The present disclosure relates to a medical image processing device and a medical observation system.

In the related art, there is known a system that generates a fluorescence image in a medical observation system that captures (observes) the inside of a living body (observation target) which is a subject (see, for example, WO 2015/156153 A).

Here, the fluorescence image is an image obtained by irradiating the observation target with excitation light (for example, near-infrared excitation light of about 750 nm to 800 nm) and capturing, by an imaging unit, fluorescence (for example, fluorescence in a wavelength band around 830 nm) from the observation target excited by the excitation light.

### SUMMARY

Meanwhile, a signal level is remarkably low in the fluorescence image because the fluorescence from the observation target is minute and the sensitivity of the imaging unit in a wavelength band of the fluorescence is also low. In addition, a surgical light used in laparoscopic surgery includes a wavelength component of near-infrared light.

Therefore, if the wavelength component of the near-infrared light included in the surgical light passes through an abdominal wall and is captured by the imaging unit in the laparoscopic surgery, the wavelength component of the near-infrared light becomes noise, which makes it difficult to discriminate a site emitting original fluorescence (a site where a fluorescent substance is present). That is, it is difficult to generate there is a problem that an image suitable for observation.

According to one aspect of the present disclosure, there is provided a medical image processing device including: fluorescence image acquisition circuitry configured to acquire a fluorescence image, obtained by irradiating an observation target with excitation light and capturing fluorescence from the observation target excited by the excitation light by an imaging unit; and a fluorescence image processor configured to execute image processing on the fluorescence image, wherein a light receiving surface of the imaging unit is provided with a color filter in which red, green, and blue filter groups having spectral characteristics different from each other are arrayed in a specific format, the fluorescence image includes red component information, green component information, and blue component information corresponding to the spectral characteristics of the red, green, and blue filter groups, respectively, and the fluorescence image processor is configured to combine the respective red component information, green component information, and blue component information for each pixel of the fluorescence image in a state where a weight of the red component information is lowered as compared with weights of the green component information and the blue component information.

### BRIEF DESCRIPTION OF THE DRAWINGS

FIG. 1 is a diagram illustrating a configuration of a medical observation system according to an embodiment;

FIG. 2 is a block diagram illustrating configurations of a camera head and a control device;

FIG. 3 is a view illustrating a color filter;

FIG. 4 is a view illustrating spectral characteristics of the color filter;

FIG. 5 is a flowchart illustrating an operation of the control device;

FIG. 6 is a view for describing the operation of the control device;

FIG. 7 is a view for describing the operation of the control device; and

FIG. 8 is a view for describing the operation of the control device.

### DETAILED DESCRIPTION

Hereinafter, modes (hereinafter, embodiments) for carrying out the present disclosure will be described with reference to the drawings. Note that the present disclosure is not limited to the embodiments to be described below. Further, the same parts are denoted by the same reference signs when the drawings are described.

Schematic Configuration of Medical Observation System

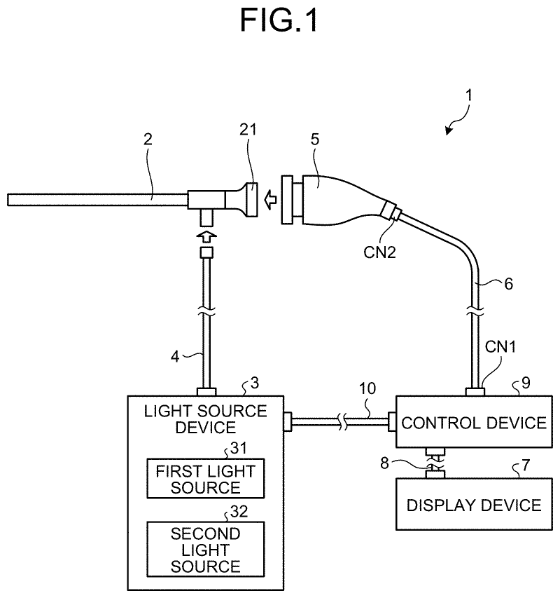

FIG. 1 is a diagram illustrating a configuration of a medical observation system 1 according to an embodiment.

The medical observation system 1 is a system that is used in the medical field and captures (observes) the inside of a living body (observation target) that is a subject. As illustrated in FIG. 1, the medical observation system 1 includes an insertion unit 2, a light source device 3, a light guide 4, a camera head 5, a first transmission cable 6, a display device 7, a second transmission cable 8, a control device 9, and a third transmission cable 10.

In the embodiment, the insertion unit 2 includes a rigid endoscope. That is, the insertion unit 2 has an elongated shape that is entirely rigid or has a part that is soft and the other part that is rigid, and is inserted into the living body. An optical system, which is configured using one or a plurality of lenses and condenses light from the subject, is provided in the insertion unit 2.

The light source device 3 is connected with one end of the light guide 4, and supplies light to the one end of the light guide 4 to irradiate the inside of the living body under control of the control device 9. As illustrated in FIG. 1, the light source device 3 includes a first light source 31 and a second light source 32.

The first light source 31 generates (emits) normal light in a visible wavelength band. In the embodiment, the first light source 31 includes a light emitting diode (LED) that emits white light. Note that the first light source 31 is not limited to the white LED, and may be configured to be capable of emitting white light by combining light emitted from each of a red LED, a green LED, and a blue LED.

The second light source 32 generates (emits) excitation light having a wavelength band different from the wavelength band of normal light. In the embodiment, the second light source 32 includes a semiconductor laser that emits near-infrared excitation light in a near-infrared wavelength band (for example, a wavelength band of about 750 nm to 800 nm). Note that the second light source 32 is not limited to the semiconductor laser, and may be configured using an LED that emits near-infrared excitation light. The near-infrared excitation light is excitation light that excites a fluorescent substance such as indocyanine green. In addition, when being excited by the near-infrared excitation light, the fluorescent substance, such as indocyanine green, emits fluorescence in a wavelength band (for example, a wavelength band around 830 nm), other than the visible range, which has a central wavelength on the longer wavelength side than a central wavelength of the wavelength band of the near-infrared excitation light. Note that the wavelength band of the near-infrared excitation light and the wavelength band of the fluorescence may be set so as to partially overlap each other, or may be set so as not to overlap each other at all.

Further, the first light source 31 is driven during a first period between alternately repeated first and second periods under control of the control device 9 in the light source device 3 according to the embodiment. That is, the light source device 3 emits normal light (white light) during the first period. In addition, the second light source 32 is driven during the second period under the control of the control device 9 in the light source device 3. That is, the light source device 3 emits near-infrared excitation light during the second period.

Note that the light source device 3 is configured separately from the control device 9 in the embodiment, but the present disclosure is not limited thereto and may adopt a configuration in which the light source device 3 is provided inside the control device 9.

The one end of the light guide 4 is detachably connected to the light source device 3, and the other end thereof is detachably connected to the insertion unit 2. Further, the light guide 4 transmits light (normal light or near-infrared excitation light) supplied from the light source device 3 from the one end to the other end, and supplies the light to the insertion unit 2. When the inside of the living body is irradiated with the normal light (white light), the normal light transmitted through the living body (normal light reflected in the living body) is condensed by the optical system in the insertion unit 2. In addition, when the inside of a living body is irradiated with the near-infrared excitation light, the near-infrared excitation light passing through the living body (near-infrared excitation light reflected in the living body) and fluorescence emitted from a fluorescent substance as the fluorescent substance, such as indocyanine green, accumulated at a lesion in the living body is excited by the near-infrared excitation light, are condensed by an optical system in the insertion unit 2.

The camera head 5 corresponds to an imaging device according to the present disclosure. The camera head 5 is detachably connected to a proximal end (an eyepiece portion 21 (FIG. 1)) of the insertion unit 2. Further, the camera head 5 captures the light condensed by the insertion unit 2 to generate a captured image under the control of the control device 9.

Note that a detailed configuration of the camera head 5 will be described in “Configuration of Camera Head” which will be described later.

The first transmission cable 6 has one end detachably connected to the control device 9 via a connector CN1 (FIG. 1), and the other end detachably connected to the camera head 5 via a connector CN2 (FIG. 1). Further, the first transmission cable 6 transmits the captured image and the like output from the camera head 5 to the control device 9, and transmits a control signal, a synchronization signal, a clock, power, and the like output from the control device 9 to the camera head 5.

Note that the captured image and the like may be transmitted as an optical signal or may be transmitted as an electrical signal in the transmission of the captured image and the like from the camera head 5 to the control device 9 via the first transmission cable 6. The same applies to the transmission of the control signal, the synchronization signal, and the clock from the control device 9 to the camera head 5 via the first transmission cable 6.

The display device 7 includes a display using liquid crystal, organic electro luminescence (EL), or the like, and displays an image based on a video signal from the control device 9 under the control of the control device 9.

The second transmission cable 8 has one end detachably connected to the display device 7 and the other end detachably connected to the control device 9. Further, the second transmission cable 8 transmits the video signal processed by the control device 9 to the display device 7.

The control device 9 corresponds to a medical image processing device according to the present disclosure. The control device 9 includes a central processing unit (CPU), a field-programmable gate array (FPGA), and the like, and integrally controls operations of the light source device 3, the camera head 5, and the display device 7.

Note that a detailed configuration of the control device 9 will be described in “Configuration of Control Device” which will be described later.

The third transmission cable 10 has one end detachably connected to the light source device 3 and the other end detachably connected to the control device 9. Further, the third transmission cable 10 transmits a control signal from the control device 9 to the light source device 3.

Configuration of Camera Head

Next, the configuration of the camera head 5 will be described.

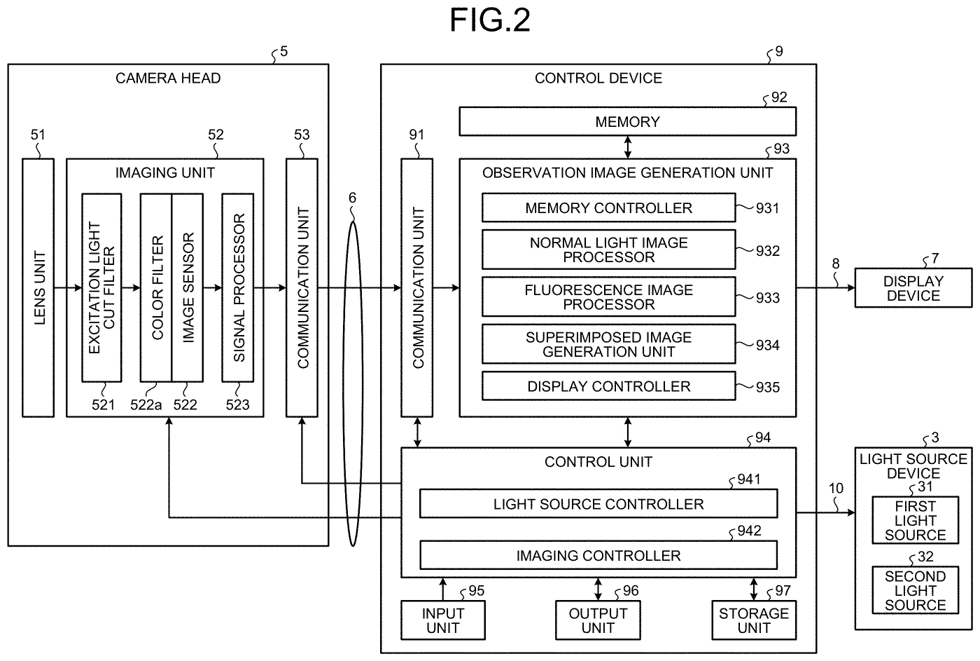

FIG. 2 is a block diagram illustrating the configurations of the camera head 5 and the control device 9.

As illustrated in FIG. 2, the camera head 5 includes a lens unit 51, an imaging unit 52, and a communication unit 53.

The lens unit 51 is configured using one or a plurality of lenses, and forms an image of light (normal light, near-infrared excitation light, or fluorescence) condensed by the insertion unit 2 on an imaging surface of the imaging unit 52 (image sensor 522).

The imaging unit 52 captures the inside of a living body under control of the control device 9. As illustrated in FIG. 2, the imaging unit 52 includes an excitation light cut filter 521, the image sensor 522, and a signal processor 523.

The excitation light cut filter 521 is provided between the lens unit 51 and the image sensor 522, and includes a band stop filter that removes a specific wavelength band. The excitation light cut filter 521 may be provided in the insertion unit 2. Note that, hereinafter, a wavelength band to be cut (removed) by the excitation light cut filter 521 will be referred to as a cut band, a wavelength band that is closer to a short wavelength side than the cut band and is transmitted through the excitation light cut filter 521 will be referred to as a short-wavelength-side transmission area, and a wavelength band that is closer to a long wavelength side than the cut band and is transmitted through the excitation light cut filter 521 will be referred to as a long-wavelength-side transmission area, for convenience of the description.

Here, the cut band includes at least a part of the wavelength band of near-infrared excitation light. In the embodiment, the cut band includes the entire wavelength band of the near-infrared excitation light. In addition, a long-wavelength-side transmission band includes the wavelength band of fluorescence. Further, the short-wavelength-side transmission area includes the wavelength band of normal light (white light).

That is, the excitation light cut filter 521 transmits normal light (white light) directed from the lens unit 51 to the image sensor 522. Note that, hereinafter, the normal light (white light) that is transmitted through the excitation light cut filter 521 and directed to the image sensor 522 will be referred to as a subject image for convenience of the description. On the other hand, the excitation light cut filter 521 removes near-infrared excitation light and transmits fluorescence for the near-infrared excitation light and the fluorescence directed from the lens unit 51 to the image sensor 522. Note that, hereinafter, the fluorescence that is transmitted through the excitation light cut filter 521 and directed to the image sensor 522 will be referred to as a fluorescent image for convenience of the description.

The image sensor 522 includes a charge coupled device (CCD), a complementary metal oxide semiconductor (CMOS), or the like that receives the subject image or the fluorescent image transmitted through the excitation light cut filter 521 and converts the received image into an electrical signal (analog signal). A color filter 522a (FIG. 2) is provided on an imaging surface (light receiving surface) of the image sensor 522.

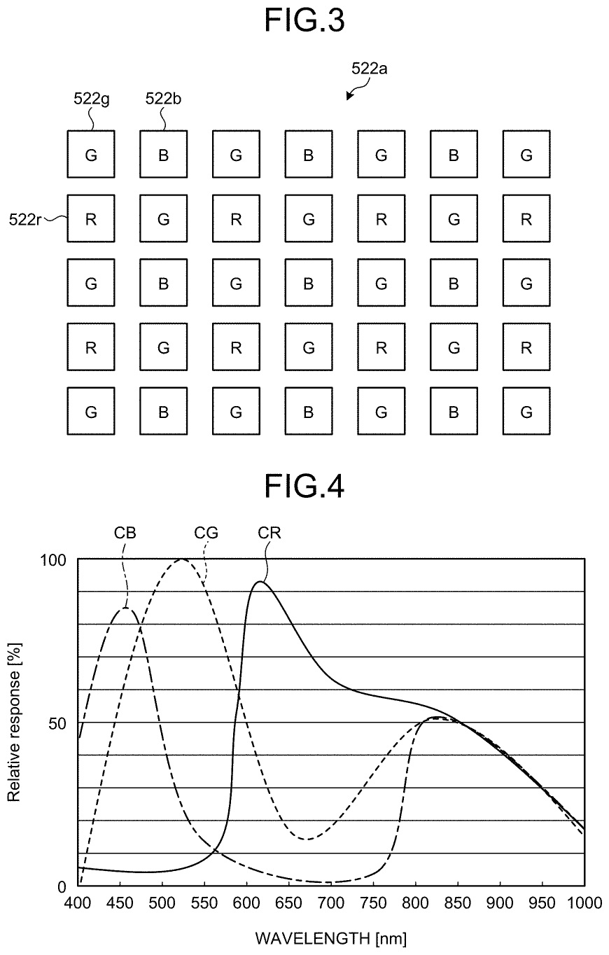

FIG. 3 is a view illustrating the color filter 522a. FIG. 4 is a view illustrating spectral characteristics of the color filter 522a. Specifically, in FIG. 4, a spectral characteristic of an R filter group 522r is indicated by a curve CR, a spectral characteristic of a G filter group 522g is indicated by a curve CG, and a spectral characteristic of a B filter group 522b is indicated by a curve CB.

The color filter 522a is a color filter in which the R, G, and B filter groups, grouped according to wavelength bands of light to be transmitted (R (red), G (green), and B (blue)), are arrayed in a specific format (for example, the Bayer array).

Specifically, as illustrated in FIGS. 3 and 4, the color filter 522a includes: the R filter group 522r (FIG. 3) that mainly transmits light in an R wavelength band; the B filter group 522b (FIG. 3) that mainly transmits light in a wavelength band of B; a first G filter group (arrayed in the same column as the R filter group 522r) that mainly transmits light in a G wavelength band; and a second G filter group (arrayed in the same column as the B filter group 522b) that mainly transmits the light in the G wavelength band. Note that the first and second G filter groups are collectively referred to as the G filter group 522g in FIG. 3. In addition, in FIG. 3, the letter “R” is attached to the R filter group 522r, the letter “G” is attached to the G filter group 522g, and the letter “B” is attached to the B filter group 522b.

As illustrated in FIG. 4, the R, G, and B filter groups 522r, 522g, and 522b have substantially the same spectral characteristics in a wavelength band of fluorescence (for example, a wavelength band around 830 nm). Further, the image sensor 522 has sensitivity not only to the light of the wavelength bands of R, G, and B but also to the wavelength band of fluorescence.

Further, the image sensor 522 performs imaging every first and second periods, which are alternately repeated, in synchronization with light emission timings of the light source device 3 under the control of the control device 9. Hereinafter, for convenience of the description, an image generated by capturing the subject image (normal light) during the first period by the image sensor 522 will be referred to as a normal light image, and an image generated by capturing the fluorescent image (fluorescence) during the second period by the image sensor 522 will be referred to as a fluorescence image. In addition, the normal light image and the fluorescence image are collectively referred to as a captured image.

The signal processor 523 performs signal processing on a captured image of an analog signal generated by the image sensor 522 and outputs a captured image of a digital signal.

The communication unit 53 functions as a transmitter that transmits the captured image output from the imaging unit 52 to the control device 9 via the first transmission cable 6. The communication unit 53 includes, for example, a high-speed serial interface that performs communication of the captured image at a transmission rate of 1 Gbps or more with the control device 9 via the first transmission cable 6.

Configuration of Control Device

Next, the configuration of the control device 9 will be described with reference to FIG. 2.

As illustrated in FIG. 2, the control device 9 includes a communication unit 91, a memory 92, an observation image generation unit 93, a control unit 94, an input unit 95, an output unit 96, and a storage unit 97.

The communication unit 91 functions as a receiver that receives a captured image output from the camera head 5 (communication unit 53) via the first transmission cable 6. The communication unit 91 includes, for example, a high-speed serial interface that performs communication of the captured image with the communication unit 53 at a transmission rate of 1 Gbps or more. That is, the communication unit 91 corresponds to a fluorescence image acquisition unit and a normal light image acquisition unit according to the present disclosure.

The memory 92 includes, for example, a dynamic random access memory (DRAM) or the like. The memory 92 may temporarily store a plurality of frames of captured images sequentially output from the camera head 5 (communication unit 53).

The observation image generation unit 93 processes the captured images sequentially output from the camera head 5 (communication unit 53) and received by the communication unit 91 under control of the control unit 94. As illustrated in FIG. 2, the observation image generation unit 93 includes a memory controller 931, a normal light image processor 932, a fluorescence image processor 933, a superimposed image generation unit 934, and a display controller 935.

The memory controller 931 controls write and readout of the captured image to and from the memory 92. More specifically, the memory controller 931 sequentially writes the captured images (normal light image and fluorescence image), sequentially output from the camera head 5 (communication unit 53) and received by the communication unit 91, into the memory 92. In addition, the memory controller 931 reads out a normal light image from the memory 92 at a specific timing, and inputs the readout normal light image to the normal light image processor 932. Further, the memory controller 931 reads out the fluorescence image from the memory 92 at a specific timing, and inputs the read fluorescence image to the fluorescence image processor 933.

The normal light image processor 932 executes first image processing on the input normal light image.

Examples of the first image processing may include optical black subtraction processing, white balance adjustment processing, demosaic processing, and color correction matrix processing, gamma correction processing, and YC processing for converting an RGB signal (normal light image) into a luminance/color difference signal (Y, Cb/Cr signal).

Here, a normal light image after having been subjected to the demosaic processing includes component information (pixel data) of R, G, and B corresponding to each of the R, G, and B filter groups 522r, 522g, and 522b, for each pixel. Hereinafter, the component information of R is described as an r value, the component information of G is described as a g value, and the component information of B is described as a b value. Further, in the YC processing, the normal light image processor 932 generates a luminance signal (Y) for each pixel by the following Formula (1).

Y=t1×r value+u1×g value+v1×b value (1)

Here, t1, u1, and v1 are values satisfying t1+u1+v1=1.

That is, the normal light image processor 932 sets weights of the respective piece of component information of R, G, and B for each pixel of the normal light image, and combines the respective pieces of component information of R, G, and B. More specifically, in Formula (1), the normal light image processor 932 may combine the respective pieces of component information of R, G, and B in a state where the weights of the respective pieces of component information of R, G, and B is the same for each pixel of the normal light image assuming that t1=0.33, u1=0.33, and v1=0.33, or may combine the respective pieces of component information of R, G, and B assuming that t1=0.3, u1=0.6, and v1=0.1.

The fluorescence image processor 933 performs second image processing different from the first image processing on the input fluorescence image.

Examples of the second image processing may include optical black subtraction processing, white balance adjustment processing, demosaic processing, and color correction matrix processing, gamma correction processing, and YC processing for converting an RGB signal (fluorescence image) into a luminance/color difference signal (Y, Cb/Cr signal), which is similar to the first image processing described above.

Here, in the YC processing executed on the fluorescence image after having been subjected to the demosaic processing, the fluorescence image processor 933 generates the luminance signal (Y) for each pixel by the following Formula (2).

Y=t2×r value+u2×g value+v2×b value (2)

Here, t2, u2, and v2 are values satisfying t2<u2, t2<v2, and t2+u2+v2=1.

That is, the fluorescence image processor 933 combines the respective pieces of component information of R, G, and B for each pixel of the fluorescence image in a state where the weight of the component information of R is lowered as compared with those of the weights of the component information of G and the component information of B. More specifically, in Formula (2), the fluorescence image processor 933 deletes the component information of R for each pixel of the fluorescence image, and combines only the component information of G and the component information of B assuming that t2=0.5, u2=0.5, and v2=0.

The superimposed image generation unit 934 executes superimposition processing for superimposing the fluorescence image on which the second image processing has been executed by the fluorescence image processor 933 on the normal light image on which the first image processing has been executed by the normal light image processor 932 to generate a superimposed image.

Here, as the superimposition processing, first and second superimposition processes to be described below may be exemplified. Note that, hereinafter, an area, which includes a pixel whose luminance value is a specific threshold or more, in a fluorescence image will be referred to as a fluorescence area.

The first superimposition process is a process of replacing an area at the same pixel position as a fluorescence area in a normal light image with an image of the fluorescence area in a fluorescence image.

The second superimposition process is a process (so-called alpha blend process) of changing brightness of a color indicating fluorescence applied to each pixel in an area at the same pixel position as a fluorescence area in a normal light image according to a luminance value at each pixel position in the fluorescence area of a fluorescence image.

The display controller 935 generates a video signal for displaying the superimposed image generated by the superimposed image generation unit 934 under the control of the control unit 94. Further, the display controller 935 outputs the video signal to the display device 7 via the second transmission cable 8.

The control unit 94 is configured using, for example, a CPU, an FPGA, or the like, and outputs a control signal via the first to third transmission cables 6, 8, and 10, thereby controlling the operations of the light source device 3, the camera head 5, and the display device 7 and controlling the overall operation of the control device 9. As illustrated in FIG. 2, the control unit 94 includes a light source controller 941 and an imaging controller 942. Note that functions of the light source controller 941 and the imaging controller 942 will be described in “Operation of Control Device” which will be described later.

The input unit 95 is configured using an operation device such as a mouse, a keyboard, and a touch panel, and receives a user operation from a user such as a doctor. Further, the input unit 95 outputs an operation signal corresponding to the user operation to the control unit 94.

The output unit 96 is configured using a speaker, a printer, or the like, and outputs various types of information.

The storage unit 97 stores a program executed by the control unit 94, information necessary for processing of the control unit 94, and the like.

Operation of Control Device

Next, the operation of the control device 9 will be described.

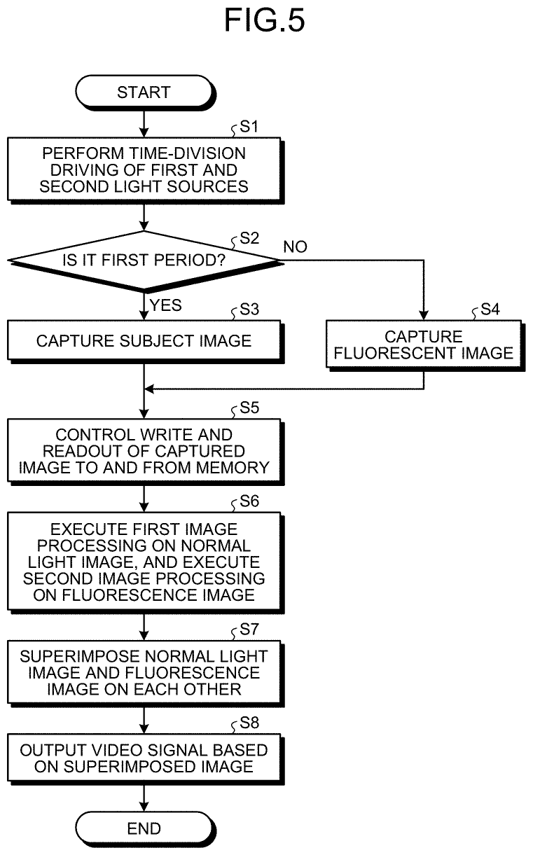

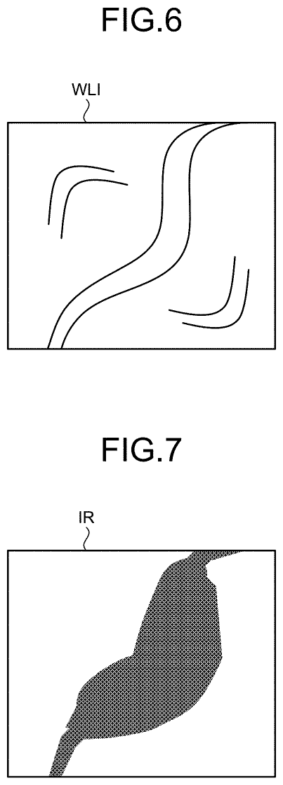

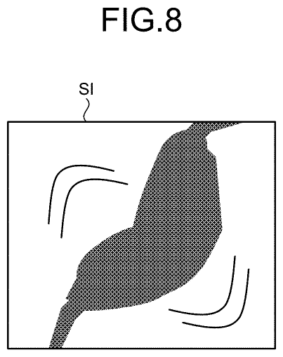

FIG. 5 is a flowchart illustrating the operation of the control device 9. FIGS. 6 to 8 are views for describing the operation of the control device 9. Specifically, FIG. 6 is the view illustrating a normal light image WLI of one frame. FIG. 7 is the view illustrating a fluorescence image IR of one frame. Note that the fluorescence image IR illustrated in FIG. 7 is expressed in gray scale, and the intensity of a captured fluorescence component is higher (luminance value is higher) as approaching black. FIG. 8 is the view illustrating a superimposed image SI of one frame generated by the superimposed image generation unit 934.

First, the light source controller 941 executes time-division driving of the first and second light sources 31 and 32 (Step S1). Specifically, in Step S1, the light source controller 941 causes the first light source 31 to emit light in a first period and causes the second light source 32 to emit light in a second period between the alternately repeated first and second periods based on a synchronization signal.

After Step S1, the imaging controller 942 causes the image sensor 522 to capture a subject image and a fluorescent image in the first and second periods in synchronization with light emission timings of the first and second light sources 31 and 32 based on the synchronization signal (Steps S2 to S4). That is, during the first period (Step S2: Yes), in other words, when the inside of a living body is irradiated with normal light (white light), the image sensor 522 captures the subject image (normal light) to generate a normal light image (Step S3). On the other hand, during the second period (Step S2: No), in other words, when the inside of the living body is irradiated with near-infrared excitation light, the image sensor 522 captures a fluorescent image (fluorescence) to generate a fluorescence image (Step S4).

After Steps S3 and S4, the memory controller 931 controls write and readout of a captured image to and from the memory 92 based on the synchronization signal (Step S5).

After Step S5, the fluorescence image processor 933 and the normal light image processor 932 execute the following processing (Step S6).

That is, the normal light image processor 932 sequentially executes first image processing on each normal light image (for example, the normal light image WLI illustrated in FIG. 6) sequentially read from the memory 92 by the memory controller 931.

In addition, the fluorescence image processor 933 sequentially executes second image processing on each fluorescence image (for example, the fluorescence image IR illustrated in FIG. 7) sequentially read from the memory 92 by the memory controller 931.

After Step S6, the superimposed image generation unit 934 executes superimposition processing for sequentially superimposing each fluorescence image (for example, the fluorescence image IR illustrated in FIG. 7) sequentially output from the fluorescence image processor 933 on each normal light image (for example, the normal light image WLI illustrated in FIG. 6) sequentially output from the normal light image processor 932 to generate a superimposed image (for example, the superimposed image SI illustrated in FIG. 8) (Step S7).

After Step S7, the display controller 935 sequentially generate a video signal for displaying each superimposed image (for example, the superimposed image SI illustrated in FIG. 8) sequentially generated by the superimposed image generation unit 934, and sequentially outputs the video signal to the display device 7 (Step S8). As a result, the superimposed image (for example, the superimposed image SI illustrated in FIG. 8) is sequentially displayed on the display device 7.

According to the embodiment described above, the following effects are achieved.

As described above, the R, G, and B filter groups 522r, 522g, and 522b have substantially the same spectral characteristics in a wavelength band of fluorescence (for example, a wavelength band around 830 nm). Further, in the control device 9 according to the present embodiment, the fluorescence image processor 933 generates the luminance signal (Y) by the above-described Formula (2) in the YC processing. That is, the fluorescence image processor 933 represents a fluorescence area by combining the respective pieces of component information of red, green, and blue in a state where the weight of the red component information is lowered as compared with those of the green component information and the blue component information for each pixel of the fluorescence image.

Therefore, with the control device 9 according to the present embodiment, noise caused by a wavelength component of near-infrared light may be reduced even in a case where the wavelength component of the near-infrared light included in a surgical light passes through an abdominal wall and is captured by the imaging unit 52 in laparoscopic surgery. That is, an image suitable for observation may be generated.

### OTHER EMBODIMENTS

The modes for carrying out the present disclosure have been described hereinbefore. However, the present disclosure is not limited only to the above-described embodiment.

In the above-described embodiment, the fluorescence image processor 933 combines the respective pieces of component information of R, G, and B for each pixel of the fluorescence image in the state where the weight of the component information of R is lowered as compared with those of the component information of G and the component information of B when executing the YC processing, but the present disclosure is not limited thereto.

For example, in white balance adjustment processing, a gain to be multiplied by each pixel value of R, G, and B is appropriately adjusted. As a result, the respective pieces of component information of R, G, and B are combined in the state where the weight of the R component information is lowered than those of the component information of G and the component information of B for each pixel of the fluorescence image.

In addition, for example, in color correction matrix processing, a color correction matrix to be multiplied by an input matrix having pixel values of R, G, and B included in the fluorescence image as matrix elements is appropriately adjusted. As a result, the respective pieces of component information of R, G, and B are combined in the state where the weight of the R component information is lowered than those of the component information of G and the component information of B for each pixel of the fluorescence image.

Meanwhile, photo-dynamic diagnosis (PDD), which is one of cancer diagnosis methods for detecting a cancer cell, is conventionally known.

In the photodynamic diagnosis, for example, a photosensitive substance such as 5-aminolevulinic acid (hereinafter, referred to as 5-ALA) is used. The 5-ALA is a natural amino acid originally contained in living organisms of animals and plants. This 5-ALA is taken into a cell after administration in vivo, and is biosynthesized into protoporphyrin in mitochondria. Further, the protoporphyrin is excessively accumulated in a cancer cell. In addition, the protoporphyrin excessively accumulated in the cancer cell has photoactivity. Therefore, the protoporphyrin emits fluorescence (for example, red fluorescence in a wavelength band of 600 nm to 740 nm) when being excited with excitation light (for example, blue visible light in a wavelength band of 375 nm to 445 nm). In this manner, a cancer diagnostic method in which a cancer cell is caused to emit fluorescence using a photosensitizer is referred to as the photo-dynamic diagnosis.

Further, the first light source 31 may be configured using an LED that emits white light and the second light source 32 may be configured using a semiconductor laser that emits excitation light for exciting protoporphyrin (for example, blue visible light in a wavelength band of 375 nm to 445 nm) in the above-described embodiment. Even in the case of adopting such a configuration, the same effects as those of the embodiment described above are obtained.

Although the first and second periods are set to be alternately repeated in the above-described embodiment, the present disclosure is not limited thereto, and at least one of the first and second periods may be continuous such that a frequency ratio between the first and second periods is a ratio other than 1:1.

The spectral characteristics of the respective filter groups constituting the color filter 522a are not limited to the spectral characteristics illustrated in FIG. 4, and color filters having other spectral characteristics may be adopted in the above-described embodiment.

Although the medical image processing device according to the present disclosure is mounted on the medical observation system 1 in which the insertion unit 2 is configured using the rigid endoscope in the above-described embodiment, the present disclosure is not limited thereto. For example, the medical image processing device according to the present disclosure may be mounted on a medical observation system in which the insertion unit 2 is configured using a flexible endoscope. In addition, the medical image processing device according to the present disclosure may be mounted on a medical observation system such as a surgical microscope (see, for example, JP 2016-42981 A) that enlarges and observes a predetermined visual field area in a subject (in a living body) or a subject surface (living body surface).

A part of the configuration of the camera head 5 or a part of the configuration of the control device 9 may be provided in, for example, the connector CN1 or the connector CN2 in the above-described embodiment.

Although the normal light image (for example, the normal light image WLI illustrated in FIG. 6) on which the first image processing has been executed and the fluorescence image (for example, the fluorescence image IR illustrated in FIG. 7) on which the second image processing has been executed are superimposed on each other to generate the superimposed image (for example, the superimposed image SI illustrated in FIG. 8) and the superimposed image is displayed on the display device 7 in the above-described embodiment, the present disclosure is not limited thereto. For example, a configuration may be adopted in which picture-in-picture processing or the like is executed, and the normal light image and the fluorescence image are simultaneously displayed on the display device 7.

Note that the following configuration also belongs to the technical scope of the present disclosure.

(1) A medical image processing device including: a fluorescence image acquisition unit that acquires a fluorescence image, obtained by irradiating an observation target with excitation light and capturing fluorescence from the observation target excited by the excitation light by an imaging unit; and a fluorescence image processor that executes image processing on the fluorescence image, in which a light receiving surface of the imaging unit is provided with a color filter in which red, green, and blue filter groups having spectral characteristics different from each other are arrayed in a specific format, the fluorescence image includes red component information, green component information, and blue component information corresponding to the spectral characteristics of the red, green, and blue filter groups, respectively, and the fluorescence image processor combines the respective red component information, green component information, and blue component information for each pixel of the fluorescence image in a state where a weight of the red component information is lowered as compared with weights of the green component information and the blue component information.

(2) The medical image processing device according to (1), in which the fluorescence image processor deletes the red component information and combines only the green component information and the blue component information for each pixel of the fluorescence image.

(3) The medical image processing device according to (1) or (2), in which the fluorescence image processor combines the respective red component information, green component information, and blue component information in a state where a weight of the red component information is lowered as compared with weights of the green component information and the blue component information when performing YC processing for generating a luminance/color difference signal from the respective red component information, green component information, and blue component information for each pixel of the fluorescence image.

(4) The medical image processing device according to any one of (1) to (3), further including: a normal light image acquisition unit that acquires a normal light image obtained by irradiating an observation target with normal light in a visible wavelength band and capturing the normal light transmitted through the observation target by the imaging unit; and a normal light image processor that executes image processing on the normal light image, in which the normal light image processor combines the respective red component information, green component information, and blue component information for each pixel of the normal light image in a state where weights of the respective red component information, green component information, and blue component information are set to be identical.

(5) The medical image processing device according to (4), in which the normal light image processor combines the respective red component information, green component information, and blue component information for each pixel of the normal light image in a state where weights of the respective red component information, green component information, and blue component information are set to be identical.

(6) A medical observation system including: an imaging device including: an imaging unit that captures fluorescence of an observation target to generate a fluorescence image, the observation target being irradiated with excitation light and excited by the excitation light; and a color filter which is provided on a light receiving surface of the imaging unit and in which red, green, and blue filter groups having spectral characteristics different from each other are arrayed in a specific format; and a medical image processing device that processes the fluorescence image, in which the medical image processing device includes: a fluorescence image acquisition unit that acquires the fluorescence image; and a fluorescence image processor that executes image processing on the fluorescence image, the light receiving surface of the imaging unit is provided with the color filter in which the red, green, and blue filter groups having the spectral characteristics different from each other are arrayed in the specific format, the fluorescence image includes red component information, green component information, and blue component information corresponding to the spectral characteristics of the red, green, and blue filter groups, respectively, and the fluorescence image processor combines the respective red component information, green component information, and blue component information for each pixel of the fluorescence image in a state where a weight of the red component information is lowered as compared with weights of the green component information and the blue component information.

With a medical image processing device and a medical observation system according to the present disclosure, it is possible to generate an image suitable for observation.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical image processing device comprising:
fluorescence image acquisition circuitry configured to acquire a fluorescence image, obtained by irradiating an observation target with excitation light and capturing fluorescence from the observation target excited by the excitation light by an imaging unit; and
a fluorescence image processor configured to execute image processing on the fluorescence image,
wherein a light receiving surface of the imaging unit is provided with a color filter in which red, green, and blue filter groups having spectral characteristics different from each other are arrayed in a specific format,
the fluorescence image includes red component information, green component information, and blue component information corresponding to the spectral characteristics of the red, green, and blue filter groups, respectively, and
the fluorescence image processor is configured to combine the respective red component information, green component information, and blue component information for each pixel of the fluorescence image in a state where a weight of the red component information is lowered as compared with weights of the green component information and the blue component information.

2. The medical image processing device according to claim 1, wherein the fluorescence image processor is configured to
delete the red component information, and
combine only the green component information and the blue component information for each pixel of the fluorescence image.

3. The medical image processing device according to claim 1, wherein the fluorescence image processor is configured to combine the respective red component information, green component information, and blue component information in a state where a weight of the red component information is lowered as compared with weights of the green component information and the blue component information when performing YC processing for generating a luminance/color difference signal from the respective red component information, green component information, and blue component information for each pixel of the fluorescence image.

4. The medical image processing device according to claim 1, further comprising:
normal light image acquisition circuitry configured to acquire a normal light image obtained by irradiating an observation target with normal light in a visible wavelength band and capturing the normal light transmitted through the observation target by the imaging unit; and
a normal light image processor configured to execute image processing on the normal light image, wherein the normal light image processor is configured to combine the respective red component information, green component information, and blue component information for each pixel of the normal light image.

5. The medical image processing device according to claim 4, wherein the normal light image processor is configured to combine the respective red component information, green component information, and blue component information for each pixel of the normal light image in a state where weights of the respective red component information, green component information, and blue component information are set to be identical.

6. A medical observation system comprising:
an imaging device including:
an imaging unit configured to capture fluorescence of an observation target to generate a fluorescence image, the observation target being irradiated with excitation light and excited by the excitation light; and
a color filter provided on a light receiving surface of the imaging unit and in which red, green, and blue filter groups having spectral characteristics different from each other are arrayed in a specific format; and
a medical image processing device configured to process the fluorescence image, wherein the medical image processing device includes:
fluorescence image acquisition circuitry configured to acquire the fluorescence image; and
a fluorescence image processor configured to execute image processing on the fluorescence image,
the fluorescence image includes red component information, green component information, and blue component information corresponding to the spectral characteristics of the red, green, and blue filter groups, respectively, and
the fluorescence image processor is configured to combine the respective red component information, green component information, and blue component information for each pixel of the fluorescence image in a state where a weight of the red component information is lowered as compared with weights of the green component information and the blue component information.
